(19) United States
(12) Patent Application Publication
(10) Pub. No.: US 2008/0031834 A1
(43) Pub. Date: Feb. 7, 2008

(54) Cosmetic Compositions

(75) Inventors: Jean Marie Manelski, Spring Lake, NJ (US); Neil D. Scancarella, Wyckoff, NJ (US); Julio Gans Russ, Westfield, NJ (US); Jodi Lorraine Kubida, Lavallette, NJ (US); Nykol Annette West, Edison, NJ (US)

Correspondence Address: JULIE BLACKBURN;REVLON CONSUMER PRODUCTS CORPORATION, 237 PARK AVENUE, NEW YORK, NY, 10017, US

(73) Assignee: Revlon Consumer Products Corporation, New York, NY (US)

(21) Appl. No.: 11/868,669

(22) Filed: Oct. 8, 2007

Related U.S. Application Data
(63) Continuation of application No. 10/389,359, filed on Mar. 15, 2003, now abandoned.

(51) Int. Cl. A61K 8/891; A61Q 17/04; A61Q 19/00; A61Q 3/00; A61Q 5/00

(52) U.S. Cl. 424/59; 424/61; 424/70.12; 424/70.7; 424/78.02

(57) ABSTRACT

A cosmetic composition comprising at least one cyclized dimethicone solvated or dispersed in a cosmetically acceptable carrier.

The subject application is a continuation of copending U.S. application Ser. No. 10/389,359, filed Mar. 15, 2003.

### BACKGROUND OF THE INVENTION

Manufacturers of cosmetic products are on an eternal quest to formulate cosmetic compositions that provide better films on keratinous surfaces. The ideal cosmetic film lasts until the consumer wants to remove it by washing with water or using remover compositions. At the same time the film provides a very natural, aesthetic appearance on the keratinous surface without looking fake or “made up”. A suitable cosmetic film should permit the underlying keratinous surface to breathe, retain moisture, and exhibit a superficially attractive appearance that is not too artificial in appearance.

Most often, polymers are incorporated into cosmetic compositions to form the cosmetic film. Generally, such polymers contain many repeating units, or monomers, that give the polymer substantive, film forming properties. Such polymers may be natural or synthetic. Natural polymers such as cellulosics, gums, and resins, have been used as film formers in cosmetics for many years. In more recent years, as polymer chemistry has advanced, polymer manufacturers have been able to manufacture a wide variety of synthetic polymers for use in cosmetics. In general, synthetic polymers fall into one of two classes: silicone polymers (based upon silicon and oxygen), or organic polymers comprised of repeating organic moieties, for example, polymers obtained by polymerizing ethylenically unsaturated monomers such as acrylates or alkylenes, optionally with organic moieties such as amides, urethanes, and the like. Certain synthetic polymers that contain both siloxane monomers and organic moieties are also known.

While synthetic polymers comprised of organic moieties such as ethylenically unsaturated monomers are excellent film formers, they sometimes do not exhibit optimal properties on keratinous surfaces such as skin. Skin is a very dynamic substrate that is in constant movement so cosmetic films that are affixed to skin or lips must exhibit some degree of plasticity. Synthetic organic polymers do not always exhibit the necessary plasticity, and will sometimes crack on dynamic keratinous surfaces such as skin. For this reason, synthetic organic polymers are not as widely used in cosmetic compositions that are applied to skin.

On the other hand, silicone polymers are excellent film formers and have been used to form cosmetic films in many successful commercial products. While silicones provide excellent wear and adhesion in general, organic synthetic polymers often provide desired surface properties that are lacking in silicones. It has been found that a certain silicone polymer, referred to as cyclized dimethicone, when used in cosmetic compositions, provides excellent substantivity to the composition, promotes formation of a suitable cosmetic film, and provides a light, pleasant feel to the composition.

It is an object of the invention to provide a cosmetic composition with excellent wear and adhesion to keratinous surfaces.

It is another object of the invention to provide a cosmetic composition that provides a composition that exhibits excellent film forming properties.

It is another object of the invention to provide a mascara that lengthens, colors, and curls lashes, and exhibits long wearing properties.

It is another object of the invention to provide a lipstick composition that is long wearing and provides a glossy finish.

It is another object of the invention to provide cosmetic compositions for application to keratinous surfaces that look natural, provide a rich color, and exhibit reduced smudging.

Another object of the invention is to provide commercially acceptable, stable, cosmetic products for making up keratinous surfaces.

### SUMMARY OF THE INVENTION

The invention comprises a cosmetic composition comprising at least one cyclized dimethicone solvated or dispersed in a cosmetically acceptable carrier.

The invention further comprises a cosmetic composition comprising at least one cyclized dimethicone and at least one non-silicone polymer in a cosmetically acceptable carrier.

The invention further comprises a cosmetic composition comprising at least one cyclized dimethicone in combination with at least one silicone polymer in a cosmetically acceptable carrier.

The invention further comprises a cosmetic composition comprising at least one cyclized dimethicone in combination with at least one polymer comprised of silicone monomers and organic monomers.

The invention further comprises a cosmetic composition comprising at least one cyclized dimethicone in a cosmetically acceptable water and oil emulsion carrier.

The invention further comprises a cosmetic composition comprising at least one cyclized dimethicone in an anhydrous cosmetically acceptable carrier.

### DETAILED DESCRIPTION

The cosmetically acceptable carrier may generally be anhydrous, or in the form of a water-in-oil or oil-in-water emulsion, the latter containing a water phase and an oil phase.

I. The Cyclized Dimethicone

The compositions of the invention contain at least one cyclized dimethicone. The term “cyclized dimethicone” means an organosiloxane comprised of repeating —[Si—O_{2/2}]—, or “D” units, which form one or more cyclized portions in the final polymer. The cyclized portions, or rings, are formed by crosslinking certain portions along the organosiloxane chain to form rings that may be structurally aligned along the polymeric chain in the manner depicted below: 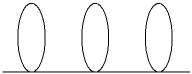
Preferably, the rings in the polymer have a molecular weight ranging from about 40,000 to 50,000, more preferably about 45,000, with the final polymer having a molecular weight ranging from about 1.6 to 2.6, preferably about 2.0 million. Cyclized dimethicone may be purchased from Jeen International under the tradename JEESILC IDD which is a mixture of cyclized dimethicone (having the INCI name dimethicone crosspolymer-3) and isododecane; or JEECHEM HPIB which is a mixture of cyclized dimethicone (dimethicone crosspolymer-3) and hydrogenated polyisbutene and cyclomethicone. The compositions of the invention may contain from about 0.1-95%, preferably about 0.5-80%, more preferably 1-75% by weight by the weight of total composition of the cyclized dimethicone.
II. Other Ingredients

The cosmetic composition in accordance with the invention may contain a variety of other ingredients including film forming polymers, pigments, waxes, oils, vitamins, and so on. Examples of such other ingredients include those described below.

A. Pigments

The composition of the invention may comprise about 0.05-30%, preferably about 0.1-25%, more preferably about 0.5-20% by weight of the total composition of one or more pigments which may be organic or inorganic. Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof. Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors which are approved for use in foods, drugs, and cosmetics by the FDA. Certified D&C and FD&C colors are listed in 21 CFR 74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 CFR 82.51. Particularly preferred are Lakes formed by the reaction of the organic pigment with a metallic salt such as aluminum, calcium, zirconium, barium, and the like. Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40. Also suitable are Lakes of such red pigments. Typically the metal salts are aluminum, barium, and the like.

Suitable yellows include those where the yellow pigment is a pyrazole, monoazo, fluoran, xanthene, quinoline, or salt thereof, such as Yellow 5, 6, 7, 8, 10, and 11, as well as Lakes of such yellow pigments.

Suitable violets include those from the anthroquinone family, such as Violet 2 and Lakes thereof. Examples of orange pigments are Orange 4, 5, 10, 11, or Lakes thereof.

Suitable inorganic pigments include iron oxides such as red, blue, black, green, and yellow; titanium dioxide, bismuth oxychloride, and the like. Preferred are iron oxides. The iron oxides may be treated with hydrophobic agents such as silicone, lecithin, mineral oil, or similar materials, will cause the pigment to be hydrophobic or lipophilic in nature, exhibiting an affinity for oily phase ingredients.

B. Particulate Fillers

The composition may contain one or more particulate fillers, which are generally non-pigmentitious powdery materials. If so, suggested ranges are about 0.001-40%, preferably about 0.05-35%, more preferably about 0.1-30% by weight of the total composition. Preferably, the particulate fillers have particle sizes ranging from about 0.02 to 100, preferably 0.5 to 100, microns. Suitable particle fillers include titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silk powder, silica, talc, mica, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof. The above mentioned powders may be surface treated with lecithin, amino acids, mineral oil, silicone oil or various other agents either alone or in combination, which coat the powder surface and render the particles more lipophilic in nature.

C. Oils

The composition may contain one or more oils, and if so in ranges from about 0.1-95%, preferably about 5-80%, more preferably about 10-75% by weight of the total composition. The term “oil” means a material that is a pourable liquid at room temperature. A variety of such oils are suitable including volatile oils, nonvolatile oils, and mixtures thereof.

1. Volatile Oils

The term “volatile” means that the oil has a measurable vapor pressure, or a vapor pressure of at least about 2 mm. of mercury at 20° C. The term “nonvolatile” means that the oil has a vapor pressure of less than about 2 mm. of mercury at 20° C. Suitable volatile oils generally have a viscosity of about 0.5 to 10 centipoise at 25° C. and include linear silicones, cyclic silicones, paraffinic hydrocarbons, or mixtures thereof.

(a). Volatile Silicones

Cyclic silicones (or cyclomethicones) are of the general formula: 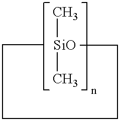
where n 3-6.

Linear volatile silicones in accordance with the invention have the general formula:
(CH₃)₃Si—O—[Si(CH₃)₂—O]ₙ—Si(CH₃)₃
where n=0-7, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, hexamethyldisiloxane, and mixtures thereof.

(b). Paraffinic Hydrocarbons

Also suitable as the volatile oil are various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 cs. at 25 degrees C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable.

2. Nonvolatile Oils

The composition may also comprise one or more non-volatile liquid oils such as silicones, esters, and the like. In the case where it is desired to make long wearing cosmetic products, if the nonvolatile oils are too heavy or greasy it may hamper the long wearing characteristics of the invention. In such a case, the viscosity of the nonvolatile oils, if present, should range from about 11-1000, preferably less than 100 centipoise, most preferably less than about 50 centipoise at 25° C. Examples of such oils include polyalkylsiloxanes, polyarylsiloxanes, and polyethersiloxanes. Examples of such nonvolatile silicones are disclosed in Cosmetics, Science and Technology 27-104 (Balsam and Sagarin ed. 1972); and U.S. Pat. Nos. 4,202,879 and 5,069,897, both of which are hereby incorporated by references. Further nonlimiting examples of such silicones include dimethicone, phenyl trimethicone, dimethicone copolyol, and so on.

Also suitable are lower viscosity organic liquids including saturated or unsaturated, substituted or unsubstituted branched or linear or cyclic organic compounds that are liquid under ambient conditions. Preferred organic liquids include those described in U.S. Pat. Nos. 5,505,937; 5,725,845; 5,019,375; and 6,214,329, all of which are incorporated by reference herein in their entirety.

If desired, the claimed composition may contain one or more nonvolatile oils. Such oils generally have a viscosity of greater than 10 centipoise at 25° C., and may range in viscosity up to 1,000,000 centipoise at 25° C. Such nonvolatile oils are preferably liquid at room temperature (e.g. 25° C.), and include those set forth below. In the event long-wearing or transfer resistant compositions are desired, if non-volatile oils are present, they are preferably of lower viscosity, e.g. ranging from about 10 to 100,000, preferably 10-50,000, more preferably 10-1000 centipoise at room temperature. Further examples of non volatile oils include those set forth below.

(a). Esters

Suitable esters are mono-, di-, and triesters. The composition may comprise one or more esters selected from the group, or mixtures thereof.

(i). Monoesters

Monoesters are defined as esters formed by the reaction of a monocarboxylic acid having the formula R—COOH, wherein R is a straight or branched chain saturated or unsaturated alkyl having 2 to 30 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups, and in one preferred embodiment of the invention the acid is an alpha hydroxy acid. Either one or both of the acid or alcohol may be a “fatty” acid or alcohol, ie. may have from about 6 to 22 carbon atoms. Examples of monoester oils that may be used in the compositions of the invention include hexyldecyl benzoate, hexyl laurate, hexadecyl isostearate, hexyldecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, cetyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

(ii). Diesters

Suitable diesters that may be used in the compositions of the invention are the reaction product of a dicarboxylic acid and an aliphatic or aromatic alcohol. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. The aliphatic or aromatic alcohol may be substituted with one or more substitutents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.

(iii). Triesters

Suitable triesters comprise the reaction product of a tricarboxylic acid and an aliphatic or aromatic alcohol. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C12-13 alkyl citrate, tricaprylin, tricaprylyl citrate, tridecyl behenate, trioctyldodecyl citrate, tridecyl behenate, tridecyl cocoate, tridecyl isononanoate, and so on.

(b). Hydrocarbon Oils.

It may be desirable to incorporate one or more non-volatile hydrocarbon oils into the claimed composition. The term “nonvolatile” means that the oil has a vapor pressure of less than about 2 mm. of mercury at 20° C.

Suitable nonvolatile hydrocarbon oils include isoparaffins and olefins having greater than 20 carbon atoms. Examples of such hydrocarbon oils include C₂₄₋₂₈ olefins, C₃₀₋₄₅ olefins, C₂₀₋₄₀ isoparaffins, hydrogenated polyisobutene, mineral oil, pentahydrosqualene, squalene, squalane, and mixtures thereof.

(c). Lanolin Oil

Also suitable for use in the composition is lanolin oil or derivatives thereof containing hydroxyl, alkyl, or acetyl groups, such as hydroxylated lanolin, isobutylated lanolin oil, acetylated lanolin, acetylated lanolin alcohol, and so on.

(d). Glyceryl Esters of Fatty Acids

The nonvolatile oil may also comprise naturally occurring glyceryl esters of fatty acids, or triglycerides. Both vegetable and animal sources may be used. Examples of such oils include castor oil, lanolin oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable as the oil are synthetic or semi-synthetic glyceryl esters, e.g. fatty acid mono-, di-, and triglycerides which are natural fats or oils that have been modified, for example, acetylated castor oil, or mono-, di- or triesters of polyols such as glyceryl stearate, diglyceryl diiosostearate, polyglyceryl-4 isostearate, polyglyceryl-6 ricinoleate, glyceryl dioleate, glyceryl diisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(e). Nonvolatile Silicones

Nonvolatile silicone oils, both water soluble and water insoluble, are also suitable for use as the non-volatile oil. Such silicones preferably have a viscosity ranging from about 10 to 600,000 centistokes, preferably 20 to 100,000 centistokes at 25° C. Suitable water insoluble silicones include amine functional silicones such as amodimethicone; phenyl substituted silicones such as bisphenylhexamethicone, phenyl trimethicone, or polyphenylmethylsiloxane; dimethicone, alkyl substituted dimethicones, and mixtures thereof.

Water soluble, non-film forming silicones such as dimethicone copolyol, dimethiconol, and the like may be used. Such silicones are available from Dow Corning as the 3225C formulation aid, Dow 190 and 193 fluids, or similar products marketed by Goldschmidt under the ABIL tradename.

Also suitable as the oil are various fluorinated oils such as fluorinated silicones, fluorinated esters, or perfluoropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers like those disclosed in U.S. Pat. Nos. 5,183,589, 4,803,067, 5,183,588 all of which are hereby incorporated by reference, which are commercially available from Montefluos under the trademark Fomblin, are also suitable shine enhancers.

(f). Fluoroguerbet Esters

Fluoroguerbet esters are also suitable oils. The term “guerbet ester” means an ester which is formed by the reaction of a guerbet alcohol having the general formula: 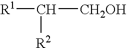
and a fluoroalcohol having the following general formula:
CF₃—(CF₂)ₙ—CH₂—CH₂—OH
wherein n is from 3 to 40.
with a carboxylic acid having the general formula:
R³COOH, or
HOOC—R³—COOH
wherein R¹, R², and R³ are each independently a straight or branched chain alkyl.

Preferably, the guerbet ester is a fluoro-guerbet ester which is formed by the reaction of a guerbet alcohol and carboxylic acid (as defined above), and a fluoroalcohol having the following general formula:
CF₃—(CF₂)ₙ—CH₂—CH₂—OH
wherein n is from 3 to 40.

Examples of suitable fluoro guerbet esters are set forth in U.S. Pat. No. 5,488,121 which is hereby incorporated by reference. Suitable fluoro-guerbet esters are also set forth in U.S. Pat. No. 5,312,968 which is hereby incorporated by reference. One type of such an ester is fluorooctyldodecyl meadowfoamate, sold under the tradename Silube GME-F by Siltech, Norcross Ga.

D. Additional Film Forming Polymers

The composition may contain one or more film forming polymers in addition to the cyclized dimethicone, and if so, ranges of about 0.1-35%, preferably 0.5-30%, more preferably 1-25% by weight of the total composition of one or more film forming polymers. The film forming polymer (or film former) may be water soluble or water insoluble. Suitable film forming polymers are those that, when the composition is applied to the desired surface, form a film on the surface to which the composition is applied when the liquid in the composition evaporates. This causes the film forming polymer to form a film which holds the other active ingredients in place with the network created by the hardened polymer. The term “soluble” means that the film forming polymer is soluble in the phase in question, and will form a single homogeneous phase when incorporated therein. For examples if the film forming polymer is oil soluble it will generally be soluble in the oil phase of the composition and when incorporated therein the oil and the polymer will form a single homogeneous phase with the oily phase ingredients. Similarly, if the film forming polymer is water soluble, if incorporated in the water phase the polymer and the water will form a single homogeneous phase. In the case where the compositions of the invention are in the emulsion form, it may also be possible for the emulsion to contain a film forming polymer that is soluble in one phase but is found dispersed in the other phase. For example, water soluble film forming polymer may be dispersed in the oil phase of the emulsion or an oil soluble polymer may be dispersed in the water phase of the emulsion. In short, any combination of film forming polymer and phase is suitable so long as the compositions are stable. The term “dispersible” means that the film forming polymer is readily dispersed in the liquid vehicle and forms a stable, heterogeneous composition where the dispersed polymer remains stable and suspended in the liquid vehicle and is compatible therewith (without settling out, for example).

A variety of film forming polymers may be suitable. Such polymers may be natural or synthetic and are further described below.

1. Synthetic Polymers

(a). Copolymers of Silicone and Organic Moieties

One type of film forming polymer that may be used in the compositions of the invention is obtained by reacting silicone moieties with ethylenically unsaturated monomers. These copolymers may be water soluble or oil soluble depending on the substituents that are found on the polymer. The resulting copolymers may be graft or block copolymers. The term “graft copolymer” is familiar to one of ordinary skill in polymer science and is used herein to describe the copolymers which result by adding or “grafting” polymeric side chain moieties (i.e. “grafts”) onto another polymeric moiety referred to as the “backbone”. The backbone may have a higher molecular weight than the grafts. Thus, graft copolymers can be described as polymers having pendant polymeric side chains, and which are formed from the “grafting” or incorporation of polymeric side chains onto or into a polymer backbone. The polymer backbone can be a homopolymer or a copolymer. The graft copolymers are derived from a variety of monomer units.

One type of polymer that may be used as the film forming polymer is a vinyl-silicone graft or block copolymer having the formula: 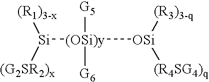
wherein G₅ represents monovalent moieties which can independently be the same or different selected from the group consisting of alkyl, aryl, aralkyl, alkoxy, alkylamino, fluoroalkyl, hydrogen, and -ZSA; A represents a vinyl polymeric segment consisting essentially of a polymerized free radically polymerizable monomer, and Z is a divalent linking group such as C₁₋₁₀ alkylene, aralkylene, arylene, and alkoxylalkylene, most preferably Z methylene or propylene.

G₆ is a monovalent moiety which can independently be the same or different selected from the group consisting of alkyl, aryl, aralkyl, alkoxy, alkylamino, fluoroalkyl, hydrogen, and -ZSA;

G₂ comprises A;

G₄ comprises A;

R₁ is a monovalent moiety which can independently be the same or different and is selected from the group consisting of alkyl, aryl, aralkyl, alkoxy, alkylamino, fluoroalkyl, hydrogen, and hydroxyl; but preferably C₁₋₄ alkyl or hydroxyl, and most preferably methyl.

R₂ is independently the same or different and is a divalent linking group such as C₁₋₁₀ alkylene, arylene, aralkylene, and alkoxyalkylene, preferably C₁₋₃ alkylene or C₇₋₁₀ aralkylene, and most preferably —CH₂— or 1,3-propylene, and

R₃ is a monovalent moiety which is independently alkyl, aryl, aralkyl, alkoxy, alkylamino, fluoroalkyl, hydrogen, or hydroxyl, preferably C₁₋₄ alkyl or hydroxyl, most preferably methyl;

R₄ is independently the same or different and is a divalent linking group such as C₁₋₁₀ alkylene, arylene, aralkylene, alkoxyalkylene, but preferably C₁₋₃ alkylene and C₇₋₁₀ alkarylene, most preferably —CH₂— or 1,3-propylene.

x is an integer of 0-3;

y is an integer of 5 or greater; preferably 10 to 270, and more preferably 40-270; and

q is an integer of 0-3.

These polymers are described in U.S. Pat. No. 5,468,477, which is hereby incorporated by reference. Most preferred is poly(dimethylsiloxane)-g-poly(isobutyl methacrylate), which is manufactured by 3-M Company under the tradename VS 70 IBM. This polymer may be purchased in the dry particulate form, or as a solution where the polymer is dissolved in one or more solvents such as isododecane. Preferred is where the polymer is in dry particulate form, and as such it can be dissolved in one or more of the liquids comprising the liquid carrier. This polymer has the CTFA name Polysilicone-6.

Another type of such a polymer comprises a vinyl, methacrylic, or acrylic backbone with pendant siloxane groups and pendant fluorochemical groups. Such polymers preferably comprise repeating A, C, D and optionally B monomers wherein:

A is at least one free radically polymerizable acrylic or methacrylic ester of a 1,1,-dihydroperfluoroalkanol or analog thereof, omega-hydridofluoroalkanols, fluoroalkylsulfonamido alcohols, cyclic fluoroalkyl alcohols, and fluoroether alcohols,

B is at least one reinforcing monomer copolymerizable with A,

C is a monomer having the general formula X(Y)nSi(R)3-m Z.m wherein

X is a vinyl group copolymerizable with the A and B monomers,

Y is a divalent linking group which is alkylene, arylene, alkarylene, and aralkylene of 1 to 30 carbon atoms which may incorporate ester, amide, urethane, or urea groups,

n is zero or 1;

m is an integer of from 1 to 3,

R is hydrogen, C₁₋₄ alkyl, aryl, or alkoxy,

Z is a monovalent siloxane polymeric moiety; and

D is at least one free radically polymerizable acrylate or methacrylate copolymer

Such polymers and their manufacture are disclosed in U.S. Pat. Nos. 5,209,924 and 4,972,037, which are hereby incorporated by reference. These polymers may be water soluble or oil soluble depending on the polymeric substituents. More specifically, the preferred polymer is a combination of A, C, and D monomers wherein A is a polymerizable acrylic or methacrylic ester of a fluoroalkylsulfonamido alcohol, and where D is a methacrylic acid ester of a C₁₋₂ straight or branched chain alcohol, and C is as defined above. Most preferred is a polymer having moieties of the general formula: has the general formula: 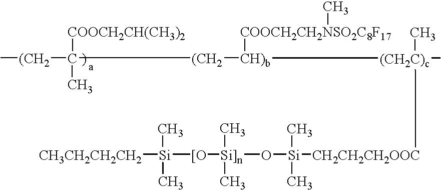
wherein each of a, b, and c has a value in the range of 1-100,000, and the terminal groups are selected from the group consisting of a C₁₋₂₀ straight or branched chain alkyl, aryl, and alkoxy and the like. These polymers may be purchased from Minnesota Mining and Manufacturing Company under the tradenames “Silicone Plus” polymers. Most preferred is poly(isobutyl methacrylate-co-methyl FOSEA)-g-poly(dimethylsiloxane) which is sold under the tradename SA 70-5 IBMMF.

Another suitable silicone acrylate copolymer is a polymer having a vinyl, methacrylic, or acrylic polymeric backbone with pendant siloxane groups. Such polymers as disclosed in U.S. Pat. Nos. 4,693,935, 4,981,903, 4,981,902, and which are hereby incorporated by reference. Preferably, these polymers are comprised of A, C, and optionally B monomers wherein:

A is at least on free radically polymerizable vinyl, methacrylate, or acrylate monomer;

B, when present, is at least one reinforcing monomer copolymerizable with A,

C is a monomer having the general formula:
X(Y)ₙSi(R)₃₋ₘZₘ
wherein:

X is a vinyl group copolymerizable with the A and B monomers;

Y is a divalent linking group;

n is zero or 1;

m is an integer of from 1 to 3;

R is hydrogen, C₁₋₁₀ alkyl, substituted or unsubstituted phenyl, C₁₋₁₀ alkoxy; and

Z is a monovalent siloxane polymeric moiety.

Examples of A monomers are lower to intermediate methacrylic acid esters of C₁₋₁₂ straight or branched chain alcohols, styrene, vinyl esters, vinyl chloride, vinylidene chloride, acryloyl monomers, and so on.

The B monomer, if present, is a polar acrylic or methacrylic monomer having at least one hydroxyl, amino, or ionic group (such as quaternary ammonium, carboxylate salt, sulfonic acid salt, and so on).

The C monomer is as above defined. These types of silicone acrylate copolymers may also be water soluble or oil soluble depending on the substituent groups on the polymer.

Most preferred is where the film forming polymer comprises Polysilicone-6, which is a dry particulate material that may be used as is or solubilized in one or more ingredients that form the liquid carrier.

Examples of other suitable copolymers that may be used herein, and their method of manufacture, are described in detail in U.S. Pat. No. 4,693,935, Mazurek, U.S. Pat. No. 4,728,571, and Clemens et al., both of which are incorporated herein by reference. Additional grafted polymers are also disclosed in EPO Application 90307528.1, published as EPO Application 0 408 311, U.S. Pat. No. 5,061,481, Suzuki et al., U.S. Pat. No. 5,106,609, Bolich et al., U.S. Pat. No. 5,100,658, Bolich et al., U.S. Pat. No. 5,100,657, Ansher-Jackson, et al., U.S. Pat. No. 5,104,646, Bolich et al., U.S. Pat. No. 5,618,524, issued Apr. 8, 1997, all of which are incorporated by reference herein in their entirety.

(b). Polymers from Ethylenically Unsaturated Monomers

Also suitable for use as film forming polymers are polymers made by polymerizing one or more ethylenically unsaturated monomers either alone or in combination with various types of organic groups, including but not limited to urethane, amides, polypropylene glycols, etc. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on. Such polymers may be water soluble or dispersible, or oil soluble or dispersible in oil.

One type of suitable polymer includes those which contain monomers which are esters of acrylic acid or methacrylic acid, including aliphatic esters of methacrylic acid like those obtained with the esterification of methacrylic acid or acrylic acid with an aliphatic alcohol of 1 to 30, preferably 2 to 20, more preferably 2 to 8 carbon atoms. If desired, the aliphatic alcohol may have one or more hydroxy groups. Also suitable are methacrylic acid or acrylic acid esters esterified with moieties containing alicyclic or bicyclic rings such as cyclohexyl or isobornyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers include those of the formula: I. 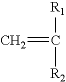
wherein R₁ is H, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or COOM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl groups, or [(CH₂)ₘO]ₙH wherein m is 1-20, and n is 1-200.

Preferably, the monofunctional ethylenically unsaturated monomer is of Formula I, above, wherein R₁ is H or a C₁₋₃₀ alkyl, and R₂ is COOM wherein M is a C₁₋₃₀ straight or branched chain alkyl which may be substituted with one or more hydroxy groups.

More preferably, R₁ is H or CH₃, and R₂ is COOM wherein 1M is a C₁₋₁₀ straight or branched chain alkyl which may be substituted with one or more hydroxy groups.

Di-, tri- and polyfunctional monomers, as well as oligomers, of the above monofunctional monomers may also be used in the composition. Suitable difunctional monomers include those having the general formula: 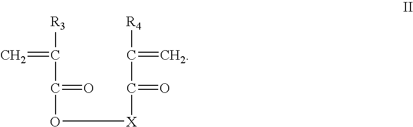
wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100. Particularly preferred are difunctional acrylates and methacrylates, such as the compound of formula II above wherein R₃ and R₄ are CH₃ and X is [(CH₂)ₓO_{y}]_{z} wherein x is 1-4; and y is 1-6; and z is 1-10.

One type of difunctional acrylate or methacrylate is the compound of formula II above wherein R₃ and R₄ are CH₃ and X is [(CH₂)ₓO_{y}]_{z} wherein x is 2; and y is 1, and z is 4. The polymerizable compositions preferably contain 0.1-25%, preferably 0.5-20%, more preferably 1-15% by weight of a difunctional monomer. Particularly preferred is where the difunctional monomer is an ethylene glycol dimethacrylate. Most preferred is where the difunctional monomer is tetraethylene glycol dimethacrylate.

Trifunctional and polyfunctional monomers are also suitable for use in the polymerizable monomer compositions of the invention. Examples of such monomers include acrylates and methacrylates such as trimethylolpropane trimethacrylate or trimethylolpropane triacrylate.

The polymers used in the compositions of the invention can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622,372; 4,656,233; 4,711,942; 4,681,918; and 4,822,859; all of which are hereby incorporated by reference.

Also suitable are polymers of Formula I, above, which are cyclized, in particular, cycloalkylacrylate polymers or copolymers having the following general formulas: 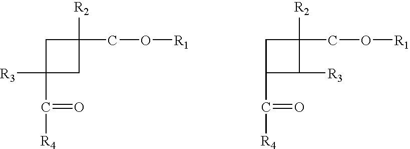
wherein R₁, R₂, R₃, and R₄ are as defined above. Typically such polymers are referred to as cycloalkylacrylate polymers. Such polymers are sold by Phoenix Chemical, Inc. under the tradename Giovarez AC-5099M. Giovarez has the chemical name isododecane acrylates copolymer and the polymer is solubilized in isododecane.

Such monomers may be copolymerized with various types of organic groups including but not limited to urethane, amide, polyalkylene glycols, and the like.

One type of organic group that can be polymerized with the above monomers includes a urethane monomer. Urethanes are generally formed by the reaction of polyhydroxy compounds with diisocyanates, as follows: 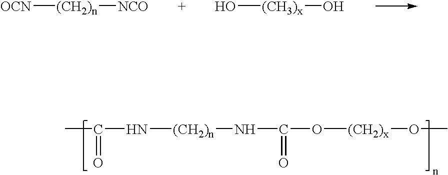
wherein n and x are each independently 1-10,000.

Another type of monomer that may be polymerized with the above comprise amide groups, preferably having the following general formula: 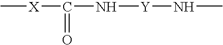
wherein X and Y are each independently linear or branched alkylene having 1-40 carbon atoms, which may be substituted with one or more amide, hydrogen, alkyl, aryl, or halogen substituents.

Another type of organic monomer may be alpha or beta pinenes, or terpenes, abietic acid, and the like.

(c). Silicone Polymers

Also suitable are various types of water soluble or water insoluble (oil soluble) high molecular weight silicone polymers such as silicone gums, resins, and the like.

Suitable silicone resins include siloxy silicate polymers having the following general formula: 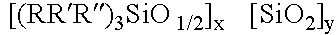
wherein R, R′ and R″ are each independently a C₁₋₁₀ straight or branched chain alkyl or phenyl, and x and y are such that the ratio of (RR′R″)₃SiO_{1/2} units to SiO₂ units is 0.5 to 1 to 1.5 to 1.

Preferably R, R′ and R″ are a C₁₋₆ alkyl, and more preferably are methyl and x and y are such that the ratio of (CH₃)₃SiO_{1/2} units to SiO₂ units is 0.75 to 1. Most preferred is this trimethylsiloxy silicate containing 2.4 to 2.9 weight percent hydroxyl groups which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. Trimethylsiloxy silicate as described is available from Dow Corning Corporation under the tradename 749 FLuid, which is a blend of about 40-60% volatile silicone and 40-60% trimethylsiloxy silicate. Dow Corning 749 fluid in particular, is a fluid containing about 50% trimethylsiloxy silicate and about 50% cyclomethicone. The fluid has a viscosity of 200-700 centipoise at 25° C., a specific gravity of 1.00 to 1.10 at 25° C., and a refractive index of 1.40-1.41. A similar siloxysilicate resin is available from GE Silicones under the tradename SR1000 and is a fine particulate solid material.

Another type of silicone resin is referred to as a T or MT resin, and has the general formula:
(R₁SiO_{3/2})x
where x ranges from about 1 to 100,000, preferably about 1-50,000, more preferably about 1-10,000, and wherein R₁ is independently C₁₋₃₀, preferably C₁₋₁₀, more preferably C₁₋₄ straight or branched chain alkyl, which may be substituted with phenyl or one or more hydroxyl groups; phenyl; alkoxy (preferably C₁₋₂₂, more preferably C₁₋₆); or hydrogen. Typically T or MT silicones are referred to as silsesquioxanes, and in the case where M units are present methylsilsesquioxanes. One type of such resin is manufactured by Wacker Chemie under the Resin MK designation. This polysilsesquioxane is a polymer comprise of T units and, optionally one or more D (preferably dimethylsiloxy) units. This particularly polymer may have ends capped with ethoxy groups, and/or hydroxyl groups, which may be due to how the polymers are made, e.g. condensation in aqueous or alcoholic media. Other suitable polysilsesquioxanes that may be used as the film forming polymer include those manufactured by Shin-Etsu Silicones and include the “KR” series, e.g. KR-220L, 242A, and so on. These particular silicone resins may contain endcap units that are hydroxyl or alkoxy groups which may be present due to the manner in which such resins are manufactured.

Another type of silicone resin suitable for use in the invention comprises the silicone esters set forth in U.S. Pat. No. 5,725,845 which is hereby incorporated by reference in its entirety. Other polymers that can enhance adhesion to skin include silicone esters comprising units of the general formula RₐR^{E}_{b}SiO_{[4-(a+b)/}2] or R¹³ₓR^{E}_{y}SiO_{1/2} wherein R and R¹³ are each independently an organic radical such as alkyl, cycloalkyl, or aryl, or, for example, methyl, ethyl, propyl, hexyl, octyl, decyl, aryl, cyclohexyl, and the like, a is a number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Preferred R_{E} radicals are those wherein the ester group is formed of one or more fatty acid moieties (e.g. of about 2, often about 3 to 10 carbon atoms) and one or more aliphatic alcohol moieties (e.g. of about 10 to 30 carbon atoms). Examples of such acid moieties include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxy)propane. Preferably the ester subgroup (i.e. the carbonyloxy radical) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety.

Preferably the silicone ester will have a melting point of no higher than about 90° C. It can be a liquid or solid at room temperature. Preferably it will have a waxy feel and a molecular weight of no more than about 100,000 Daltons.

Silicone esters having the above formula are disclosed in U.S. Pat. No. 4,725,658 and U.S. Pat. No. 5,334,737, which are hereby incorporated by reference. Preferred silicone esters are the liquid siloxy silicates disclosed in U.S. Pat. No. 5,334,737, e.g. diisostearoyl trimethylolpropane siloxysilicate (prepared in Examples 9 and 14 of this patent), and dilauroyl trimethylolpropane siloxy silicate (prepared in Example 5 of the patent), which are commercially available from General Electric under the tradenames SF 1318 and SF 1312, respectively.

Silicone gums or other types of silicone solids may be used provided they are soluble in the liquid vehicle. Examples of silicone gums include those set forth in U.S. Pat. No. 6,139,823, which is hereby incorporated by reference. Preferred gums have a 600,000 to 1,000,000 centipoise at 25° C.

2. Natural Polymers

Also suitable for use are one or more naturally occurring water soluble or oil soluble polymeric materials such as resinous plant extracts including such as rosin, shellac, and the like.

E. Plasticizers

It may be desirable to incorporate one more plasticizers into the composition. Plasticizers may improve the spreadability and application of the composition to the surface to which it is applied and in some cases will interact with the film forming polymer to make it more flexible. If present, the plasticizer may be found in the oil or water phase if the composition of the invention is in the form of an emulsion, and in the oil or lipophilic phase if the composition is in the anhydrous form. Suggested ranges of plasticizers range from about 0.01-20%, preferably about 0.05-15%, more preferably about 0.1-10% by weight of the total composition. A variety of plasticizers are suitable including Suitable plasticizers include glyceryl, glycol, and citrate esters as disclosed in U.S. Pat. No. 5,066,484, which is hereby incorporated by reference. Examples of such esters include glyceryl tribenzoate, glyceryl triacetate, acetyl tributyl citrate, dipropylene glycol dibenzoate, and the like. Also suitable, are plasticizers of the following general formula: 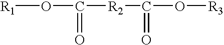
wherein R₁, R₂, and R₃ are each independently a C₁₋₂₀ straight or branched chain alkyl or alkylene which may be substituted with one or more hydroxyl groups. Preferably, R₁ is a C₃₋₁₀ straight or branched chain alkyl; R₂ is a C₂₋₈ alkyl, which may be substituted with one or more hydroxyl groups; and R₃ is a C₃₋₁₀ straight or branched chain alkyl. Examples of such compounds include dioctyl malate, diisopropyl adipate, dibutyl adipate, dibutyl sebacate, dioactyl azelate, dioctyl succinate, dioctyl fumarate, and the like.

F. Viscosity Modifiers

It may also be desirable to include one or more viscosity modifiers or thickeners in the composition. Suggested ranges of such viscosity modifiers are about 0.01-60%, preferably about 0.05-50%, more preferably about 0.1-45% by weight of the total composition.

One type of viscosity modifier includes natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like. Particularly preferred is Quaternium-18 hectorite.

Also suitable as the viscosity modifier are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonyphenyl

Another type of viscosity modifier that may be used in the compositions are silicas, silicates, silica silylate, and derivatives thereof. These silicas and silicates are generally found in the particulate form. Particularly preferred is silica.

The viscosity modifiers may also be water soluble or water insoluble (e.g. oil soluble) and form part of the oil phase or the water phase.

Also suitable as viscosity modifiers are one or more waxes. A variety of waxes are suitable including animal, vegetable, mineral, or silicone waxes. Generally such waxes have a melting point ranging from about 28 to 125° C., preferably about 30 to 100° C. Examples of waxes include acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax.

Also suitable are various types of silicone waxes, referred to as alkyl silicones, which are polymers that comprise repeating dimethylsiloxy units in combination with one or more methyl-long chain alkyl siloxy units wherein the long chain alkyl is generally a fatty chain that provides a wax-like characteristic to the silicone. Such silicones include, but are not limited to stearoxydimethicone, behenoxy dimethicone, stearyl dimethicone, cetearyl dimethicone, and so on. Suitable waxes are set forth in U.S. Pat. No. 5,725,845, which is hereby incorporated by reference in its entirety. Preferred ranges of wax are from about 0.01-75%, preferably about 1-65% by weight of the total composition.

G. Surfactants

The compositions of the invention may comprise about 0.01-20%, preferably about 0.1-15%, more preferably about 0.5-10% by weight of the total composition of a surfactant. Surfactants may be used in both anhydrous and emulsion based compositions. The surfactant may be nonionic, although if the composition is in the form of a shampoo or conditioner it will preferably contain anionic or cationic surfactants, respectively.

Suitable nonionic surfactants or emulsifiers include alkoxylated alcohols, or ethers, formed by the reaction of an alcohol with an allylene oxide, usually ethylene or propylene oxide. Preferably the alcohol is a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Preferred are alkoxylated alcohols which are formed by the reaction of stearic acid, methyl glucose, and ethoxylated alcohol, otherwise known as PEG-20 methyl glucose sesquiisostearate.

Also suitable as the nonionic surfactant are alkyoxylated carboxylic acids, which are formed by the reaction of a carboxylic acid with an alkylene oxide or with a polymeric ether. The resulting products have the general formula: 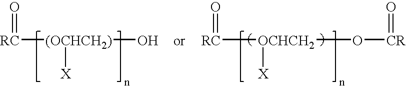
where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

Also suitable as the nonionic surfactant are monomeric, homopolymeric and block copolymeric ethers. Such ethers are formed by the polymerization of monomeric alkylene oxides, generally ethylene or propylene oxide. Such polymeric ethers have the following general formula: 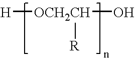
wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

Also suitable as nonionic surfactants are silicone surfactants, which are defined as silicone polymers, which have at least one hydrophilic radical and at least one lipophilic radical. The silicone surfactant used in the compositions of the invention are organosiloxane polymers that may be a liquid or solid at room temperature. The organosiloxane surfactant is generally a water-in-oil or oil-in-water type surfactant which is, and has an Hydrophile/Lipophile Balance (HLB) of 2 to 18. Preferably the organosiloxane is a nonionic surfactant having an HLB of 2 to 12, preferably 2 to 10, most preferably 4 to 6. The HLB of a nonionic surfactant is the balance between the hydrophilic and lipophilic portions of the surfactant and is calculated according to the following formula:
HLB=7+11.7×log M_{w}/Mₒ
where M_{w} is the molecular weight of the hydrophilic group portion and Mₒ is the

Examples of silicone surfactants are those sold by Dow Corning under the tradename Dow Corning 3225C Formulation Aid, Dow Corning 190 Surfactant, Dow Corning 193 Surfactant, Dow Corning Q2-5200, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention. Such types of silicone surfactants are generally referred to as dimethicone copolyols or alkyl dimethicone copolyols.

Suitable cationic, anionic, zwitterionic, and amphoteric surfactants are disclosed in U.S. Pat. No. 5,534,265, which is hereby incorporated by reference in its entirety.

H. Sunscreens

If desired, the compositions of the invention may contain 0.001-20%, preferably 0.01-10%, more preferably 0.05-8% of one or more sunscreens. A sunscreen is defined as an ingredient that absorbs at least 85 percent of the light in the UV range at wavelengths from 290 to 320 nanometers, but transmits UV light at wavelengths longer than 320 nanometers. Sunscreens generally work in one of two ways. Particulate materials, such as zinc oxide or titanium dioxide, as mentioned above, physically block ultraviolet radiation. Chemical sunscreens, on the other hand, operate by chemically reacting upon exposure to UV radiation. Suitable sunscreens that may be included in the compositions of the invention are set forth on page 582 of the CTFA Cosmetic Ingredient Handbook, Second Edition, 1992, as well as U.S. Pat. No. 5,620,965, both of which are hereby incorporated by reference. Further examples of chemical and physical sunscreens include those set forth below.

1. UVA Chemical Sunscreens

The term “UVA sunscreen” means a chemical compound that blocks UV radiation in the wavelength range of about 320 to 400 nm. Preferred UVA sunscreens are dibenzoylmethane compounds having the general formula: 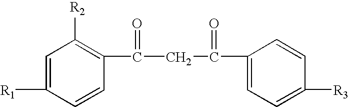
wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

The claimed compositions may contain from about 0.001-20%, preferably 0.005-5%, more preferably about 0.005-3% by weight of the composition of UVA sunscreen. In one preferred embodiment of the invention the UVA sunscreen is Avobenzone, and it is present at not greater than about 3% by weight of the total composition.

2. UVB Chemical Sunscreens

The term “UVB sunscreen” means a compound that blocks UV radiation in the wavelength range of from about 290 to 320 nm. A variety of UVB chemical sunscreens exist including α-cyano-β,β-diphenyl acrylic acid esters as set forth in U.S. Pat. No. 3,215,724, which is hereby incorporated by reference in its entirety. Particularly preferred is Octocrylene, which is 2-ethylhexyl 2-cyano-3,3-diphenylacrylate. Preferred is where the composition contains no more than about 10% by weight of the total composition of octocrylene. Suitable amounts range from about 0.001-10% by weight Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula: 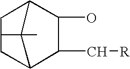
wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula: 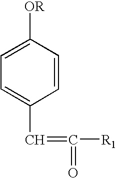
wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at nor more than about 3% by weight of the total composition.

Also suitable as the UVB screening agents are various benzophenone derivatives having the general formula: 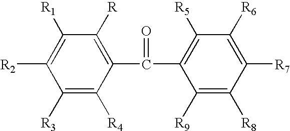
R through R₉ are each independently H, OH, NaO₃S, SO₃H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone) and Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula: 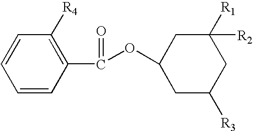
wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomethyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula: 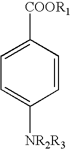
Wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: 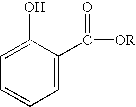
wherein R is a straight or branched chain alkyl, including derivatives of the above compound formed from mono-, di-, or triethanolamines. Particular preferred are octyl salicylate, TEA-salicylcate, DEA-salicylate, and mixtures thereof.

Generally, the amount of the UVB chemical sunscreen present may range from about 0.001-45%, preferably 0.005-40%, more preferably about 0.01-35% by weight of the total composition.

3. Physical Sunscreens

The composition may also contain one or more physical sunscreens. The term “physical sunscreen” means a material that is generally particulate in form that is able to block UV rays by forming an actual physical block on the skin. Examples of particulates that serve as solid physical sunblocks include titanium dioxide, zinc oxide and the like in particle sizes ranging from about 0.001-50 microns, preferably less than 1 micron.

J. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophosphate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

K. Humectants

If desired, the compositions of the invention comprise about 0.01-30%, preferably about 0.5-25%, more preferably about 1-20% by weight of the total composition of one or more humectants. Suitable humectants include di- or polyhydric alcohols such as glycols, sugars, and similar materials. Suitable glycols include alkylene glycols such as propylene, ethylene, or butylene glycol; or polymeric alkylene glycols such as polyethylene and polypropylene glycols, including PEG 4-240, which are polyethylene glycols having from 4 to 240 repeating ethylene oxide units. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on.

L. Other Botanical Extracts

It may be desirable to include one or more additional botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including acacia (dealbata, farnesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2.

M. Water Soluble Gellants

If the composition is in the emulsion form, it may be desirable to include other water soluble gellants in the water phase of the composition to provide thickening. Such gellants may be included a range of about 0.1-20%, preferably about 1-18%, more preferably about 2-10% by weight of the total composition is suggested, if present. Suitable gellants include soaps, i.e. salts of water insoluble fatty acids with various bases. Examples of soaps include the aluminum, calcium, magnesium, potassium, sodium, or zinc salts of C₆₋₃₀, preferably C₁₀₋₂₂ fatty acids.

Also suitable are hydrocolloids such as gellan gum, gum arabic, carrageenan, and those set forth in U.S. Pat. No. 6,197,319 which is hereby incorporated by reference in its entirety.

N. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

O. Emulsion Stabilizers

If the composition of the invention is in the emulsion form, it may be desirable to incorporate one or more emulsion stabilizers in the composition. If so, suggested ranges are about 0.0001-5%, preferably about 0.0005-3%, more preferably about 0.001-2% by weight of the total composition. Suitable emulsion stabilizers include salts of alkali or alkaline earth metal chlorides or hydroxides, such as sodium chloride, potassium chloride, and the like.

III. Forms of the Cosmetic Compositions

The cosmetic compositions in accordance with the invention may be in a variety of forms include any type of cosmetic composition applied to keratinous surfaces for the purpose of coloring, conditioning, or otherwise beautifying the keratinous surface.

A. Foundation Makeup, Color Cosmetics

Foundation makeup or color cosmetics such as eyeshadow, blush, concealer, or eyeliner compositions in the liquid, cream, solid, or stick form. Suitable foundation makeup compositions may be water-in-oil or oil-in-water emulsions. Such compositions generally comprise about:

0.001-90% cyclized dimethicone,

0.5-95% water,

0.5-25% particulate matter,

0.01-20% surfactant, and

0.1-95% nonvolatile or volatile oil.

In addition, these composition may further contain ingredients selected from the group of humectants, preservatives, gellants, and all of the ingredients as set forth above in the ranges set forth herein.

Various anhydrous color cosmetic products may also be suitable, such as blush, powder, lipsticks, eyeshadows, and the like. Such anhydrous color cosmetic compositions may generally comprise about:

0.001-80% cyclized dimethicone,

0.1-99% oil,

0.1-80% particulate matter; and optionally

0.001-50% thickening agent.

The compositions may additionally contain the various other ingredients set forth above and in the ranges taught.

Preferably, the compositions are in the form of a lipcolor or lipstick which may be a composition for coloring the lips that is in liquid, semi-solid, or solid form.

Alternatively, the composition may be in the form of a base lip color, which is a lip color applied to the lips as a basecoat to provide color, followed by application of a separate gloss coat which comprises one or more oils or waxes that provide shine, moisturization, or similar benefits to the layers applied to the lips. Examples of such lip compositions and topcoats are disclosed in U.S. Patent Application No. 2002/0159960, entitled “Method for Improving the Properties of Transfer Resistant Lip Compositions and Related Compositions and Articles”, claiming priority from provisional application No. 60/271,849, filed Feb. 27, 2001; which is hereby incorporated by reference in its entirety.

B. Lotions, Creams, Gels, and Sunscreens

The cosmetic compositions of the invention may be in the form of lotions, gels or sunscreens. Suitable skin care lotions and creams are in the emulsion form, and may be water-in-oil or oil-in-water emulsions, preferably oil-in-water emulsions. Creams, lotions, and/or may contain the following ranges of ingredients:

about 0.001-80% of the cyclized dimethicone,

about 0.1-90% oil, and

about 0.01-20% surfactant.

C. Skin and Hair Cleansing and Conditioning Compositions

Skin and hair cleansing and conditioning compositions such as facial cleansers, shampoos, hair conditioners and the like are also suitable cosmetic compositions in accordance with the invention.

Generally skin and hair cleansing compositions comprise about:

0.001-90% of the cyclized dimethicone,

1-95% water, and

0.1-40% surfactant, preferably an anionic, amphoteric, or zwitterionic surfactant.

0.01-40% oil.

Suitable hair conditioner compositions comprise:

0.001-80% of the cyclized dimethicone,

0.1-20% cationic surfactant,

0.1-30% fatty alcohol,

0.001-10% nonionic surfactant, and

5-95% water.

Suitable cationic and nonionic surfactants are as mentioned herein. Examples of suitable fatty alcohols include those having the general formula R—OH, wherein R is a C₆₋₃₀ straight or branched chain, saturated or unsaturated alkyl.

D. Nail Enamel Compositions

The cosmetically acceptable carrier for use may also comprise nail enamel compositions. Such compositions generally comprise:

0.001-90% of the cyclized dimethicone,

0.01-80% solvent,

0.001-40% particulate matter, and

optionally 0.01-40% of one or more polymers such as cellulosic polymers, acrylate polymers, and the like.

Suitable solvents include acetone, alkyl acetates such as ethyl acetate butyl acetate and the like, alkyl ethers such as propylene glycol monomethyl ether, and the like.

The invention will be further described in connection with the following examples which are set forth for the purposes of illustration only.

### EXAMPLE 1

An emulsion mascara composition was prepared as follows:

| | w/w % |
|---|---|
| Acacia Senegal gum | 1.75 |
| Triethanolamine | 2.25 |
| Lecithin/polysorbate 20/sorbitan laurate/propylene | 0.20 |
| glycol stearate/propylene glycol laurate | |
| Simethicone | 0.20 |
| Hydroxyethylcellulose | 0.20 |
| Panthenol | 0.50 |
| Nylon-12 | 1.50 |
| Methylparaben | 0.30 |
| Polyethylene | 0.80 |
| Iron oxides | 9.00 |
| Polysilicone 6 | 3.00 |
| Isododecane | 5.00 |
| Nylon 611/dimethicone copolymer/PPG-3 myristyl ether | 2.00 |
| Stearic acid | 5.60 |
| Paraffin | 10.80 |
| Beeswax | 2.80 |
| Glyceryl stearate | 2.30 |
| Phenoxyethanol | 1.00 |
| Propyl paraben | 0.10 |
| Carnauba wax | 3.50 |
| Cyclomethicone/dimethiconol | 2.70 |
| Cyclized dimethicone (dimethicone crosspolymer-3) | 0.30 |
| hydrogenated polyisobutene/cyclomethicone | |
| Phytantriol | 0.40 |
| Polyglycery-3 distearate/polysorbate 60/myristic acid/ | 0.60 |
| palmitic acid/guar hydroxypropyltrimonium | |
| chloride/tritcum vulgare (wheat) flour lipids/avocado | |
| oil | |
| Water | QS100 |

The composition was prepared by combining the water soluble pigments and water phase and mixing well. The remaining oil phase ingredients were separately mixed. Both phases were combined and emulsified to form the final composition, which was a eyelash color in a rich black shade.

### EXAMPLE 2

A lipstick composition was prepared according to the following formula:

| | w/w % |
|---|---|
| Dow Corning 2-8178, cyclized dimethicone | 15.20 |
| Isostearyl alcohol | 18.40 |
| Isododecane | 44.70 |
| Trioctyldodecyl citrate | 13.70 |
| Pigments and mica | 8.00 |

The composition was prepared by combining the ingredients with heat and mixing well.

### EXAMPLE 3

A long wearing foundation makeup composition in the emulsion form was prepared as follows:

| | w/w % |
|---|---|
| Cyclomethicone/dimethicone copolyol | 19.50 |
| Sorbitan sesquioleate | 0.05 |
| Propyl paraben | 0.10 |
| Titanium dioxide/methicone | 8.00 |
| Silk powder | 0.10 |
| Mica/methicone | 1.21 |
| Iron oxides/methicone/boron nitride | 1.00 |
| Iron oxides/methicone | 1.29 |
| Nylon-12 | 2.00 |
| Boron nitride | 3.51 |
| Dimethicone | 1.00 |
| Trimethylsiloxysilicate | 5.00 |
| Cyclomethicone | 9.00 |
| Cetyl dimethicone copolyol | 0.25 |
| Bisabolol | 0.05 |
| Tribehenin | 1.50 |
| Nylon-611/dimethicone copolymer/PPG-3 myristyl | 0.50 |
| ether | |
| Glyceryl rosinate in isododecane (44:56) | 4.50 |
| Cyclized dimethicone/hydrogenated polyisobutene | 2.25 |
| cyclomethicone | |
| Sodium chloride | 1.00 |
| Tetrasodium EDTA | 0.01 |
| Butylene glycol | 4.50 |
| Methylparaben | 0.20 |
| SD-alcohol 40B | 3.00 |
| Ethylene brassylate | 0.35 |
| Tocopheryl acetate | 0.10 |
| Retinyl palmitate | 0.05 |
| Water | QS to 100 |

The composition was prepared by combining the water phase ingredients. Separately the oil phase ingredients were combined. The two phases were combined and mixed well to emulsify. The resulting foundation makeup was poured into bottles.

### EXAMPLE 4

A lip gloss composition is made as follows:

| | w/w % |
|---|---|
| Triisostearyl citrate | 22.2 |
| Diiosostearyl malate | 7.4 |
| Octyldodecanol | 8.1 |
| Trioctyldodecyl citrate | 1.5 |
| Phenyl trimethicone | 6.2 |
| Polysilicone-6 | 12.3 |
| Cyclized dimethicone/hydrogenated polyisobutene | 2.5 |
| cyclomethicone | |
| Methylparaben | 0.4 |
| Propyl paraben | 0.2 |
| BHT | 0.1 |
| Benzoic acid | 0.2 |
| Isododecane | 6.2 |
| Polybutene | 12.3 |
| Mica/titanium dioxide | 7.1 |
| Mica/iron oxides/titanium dioxide | 1.4 |
| Mica | 4.8 |
| Pigments | 4.3 |

The composition is prepared by combining the ingredients with heat and mixing well. The resulting composition is a colored semi-solid.

### EXAMPLE 5

A face cream in the water and oil emulsion form is prepared as follows:

| | w/w % |
|---|---|
| Glycerin | 5.0 |
| Xanthan gum | 0.3 |
| Trisodium EDTA | 0.05 |
| Aloe Barbadensis leaf juice | 0.50 |
| Methylparaben | 0.25 |
| Butylene glycol | 1.00 |
| Magnesium aluminum silicate | 1.00 |
| Magnesium ascorbyl phosphate | 0.20 |
| Phenyl trimethicone | 3.00 |
| Tocopheryl acetate | 1.00 |
| Butylene glycol dicaprylate/dicaprate | 9.00 |
| Dimethicone | 1.00 |
| C12-15 alkyl benzoate | 5.00 |
| Propylparaben | 0.10 |
| Phenoxyethanol | 1.00 |
| Cetyl alcohol | 4.00 |
| Cyclized dimethicone/hydrogenated | 2.00 |
| polyisobutene cyclomethicone | |
| Tetrahexyldecyl ascorbate | 1.00 |
| Glyceryl stearate/stearic acid/cetearyl | 5.00 |
| alcohol/palmitoyl hydrolyzed | |
| wheat protein | |
| Water | QS 100 |

The composition is prepared by combining the oil phase and water phase ingredients separately, then mixing well to emulsify. The composition is of a creamy consistency.

### EXAMPLE 6

A sunscreen composition is prepared as follows:

| | w/w % |
|---|---|
| Glycerin | 5.0 |
| Xanthan gum | 0.3 |
| Trisodium EDTA | 0.05 |
| Aloe Barbadensis leaf juice | 0.50 |
| Methylparaben | 0.25 |
| Butylene glycol | 1.00 |
| Magnesium aluminum silicate | 1.00 |
| Magnesium ascorbyl phosphate | 0.20 |
| Phenyl trimethicone | 3.00 |
| Tocopheryl acetate | 1.00 |
| Butylene glycol dicaprylate/dicaprate | 9.00 |
| Dimethicone | 1.00 |
| C12-15 alkyl benzoate | 5.00 |
| Propylparaben | 0.10 |
| Phenoxyethanol | 1.00 |
| Cetyl alcohol | 4.00 |
| Cyclized dimethicone/hydrogenated | 2.00 |
| polyisobutene cyclomethicone | |
| Oxybenzone | 2.00 |
| Octinoxate | 7.50 |
| Tetrahexyldecyl palmitate | 1.00 |
| Glyceryl stearate/stearic acid/cetearyl | 5.00 |
| alcohol/palmitoyl hydrolyzed | |
| wheat protein | |
| Water | QS 100 |

The sunscreen composition is prepared by combining the oil phase and water phase ingredients separately, then combining and mixing well to emulsify.

### EXAMPLE 7

A liquid composition suitable for use as eyeliner was made as follows:

| | w/w % |
|---|---|
| Isododecane | 19.60 |
| Nylon 611/dimethicone copolymer/ | 5.00 |
| PPG-3 myristyl ether | |
| Polysilicone-6 | 25.00 |
| Blue 1 lake | 4.00 |
| Red 40 lake | 3.60 |
| Yellow 5 lake | 0.80 |
| Green 5 | 0.05 |
| Silica | 7.00 |
| Isododecane/quaternium-18 hectorite | 25.80 |
| propylene carbonate | |
| Dibutyl adipate | 2.95 |
| Methylparaben | 0.35 |
| Dehydroacetic acid | 0.20 |
| Propyl paraben | 0.10 |
| Sorbic acid | 0.06 |
| Dimethicone crosspolymer-3/ | 5.50 |
| isododecane | |

### EXAMPLE 8

A makeup remover composition was prepared as follows:

| | w/w % |
|---|---|
| Butylene glycol dicaprylate/dicaprate | 10.00 |
| Dimethicone crosspolymer-3/isododecane | 5.00 |
| Phenoxyethanol | 1.00 |
| Propylparaben | 0.10 |
| Isododecane/quaternium-18 hectorite/ | 20.00 |
| propylene carbonate | |
| Cetyl dimethicone copolyol | 2.50 |
| Cyclomethicone | 1.14 |
| Water | 55.00 |
| Butylene glycol | 5.00 |
| Trisodium EDTA | 0.01 |
| Methylparaben | 0.25 |

The composition is prepared by separately combining the oil phase ingredients and the water phase ingredients, then mixing well to emulsify.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A cosmetic composition comprising dimethicone crosspolymer-3 having a molecular weight of about 1.6 to about 2.6 million and at least one silicone monomer, organic monomer, or mixtures thereof.

2. The composition of claim 1, wherein the composition contains at least one surfactant.

3. The composition of claim 1, wherein the dimethicone crosspolymer-3 comprises a siloxane backbone with cyclized portions formed by crosslinking portions along the polymeric siloxane backbone, wherein the cyclized portions have a molecular weight of about 40,000 to about 50,000.

4. The composition of claim 1, wherein the composition optionally contains one or more of a pigment, a particulate filler, an oil, a plasticizer, a viscosity modifier, a sunscreen, a vitamin, an antioxidant, a humectant, a botanical extract, a gallant, a preservative, and an emulsion stabilizer alone or in combination.

5. The composition of claim 1, wherein the composition optionally contains one or more film forming polymer.

6. The composition of claim 1, wherein the composition is a mascara, a lipgloss, a foundation, a lipstick, a sunscreen, an eyeliner, and a makeup remover,

7. The composition of claim 1, wherein the composition is an eyeshadow, a blush, a concealer, a lotion, cream, gel, skin cleansing product, a hair cleansing product, a hair conditioning product, and a nail enamel.

8. The composition of claim 4, wherein the composition comprises one or volatile components of about 1-95% by weight of the total composition.

9. A cosmetic composition comprising dimethicone crosspolymer-3 having a molecular weight of about 1.6 to about 2.6 million, and one or more film forming polymers, polybutene, and hydrogenated polyisobutene alone or in combination.

10. The cosmetic composition of claim 9, wherein the film forming polymer is ethylene/propylene/styrene copolymer.

11. The composition of claim 9, wherein the composition optionally contains one or more of a pigment, a particulate filler, an oil, a plasticizer, a viscosity modifier, a surfactant, a sunscreen, a vitamin, an antioxidant, a humectant, a botanical extract, a gallant, a preservative, and an emulsion stabilizer alone or in combination.

12. The composition of claim 9, wherein the composition is a mascara, a lipgloss, a foundation, a lipstick, a sunscreen, an eyeliner, and a makeup remover,

13. The composition of claim 9, wherein the composition is an eyeshadow, a blush, a concealer, a lotion, cream, gel, skin cleansing product, a hair cleansing product, a hair conditioning product, and a nail enamel.

14. A cosmetic composition comprising dimethicone crosspolymer-3 having a molecular weight of about 1.6 to about 2.6 million, and one or more film forming polymers, polybutene, and hydrogenated polyisobutene alone or in combination, provided the composition does not contain a wax or a silicone.

15. The cosmetic composition of claim 14, wherein the film forming polymer is ethylene/propylene/styrene copolymer.

16. The composition of claim 14, wherein the composition optionally contains one or more of a pigment, a particulate filler, an oil, a plasticizer, a viscosity modifier, a surfactant, a sunscreen, a vitamin, an antioxidant, a humectant, a botanical extract, a gallant, a preservative, and an emulsion stabilizer alone or in combination.

17. The composition of claim 14, wherein the composition is a mascara, a lipgloss, a foundation, a lipstick, a sunscreen, an eyeliner, and a makeup remover,

18. The composition of claim 14, wherein the composition is an eyeshadow, a blush, a concealer, a lotion, cream, gel, skin cleansing product, a hair cleansing product, a hair conditioning product, and a nail enamel.

19. The composition of claim 1, wherein the composition contains a cyclized dimethicone/hydrogenated polyisobutene cyclomethicone.

20. The composition of claim 9, wherein the composition contains a cyclized dimethicone/hydrogenated polyisobutene cyclomethicone.
